(19) United States
(12) Patent Application Publication
(10) Pub. No.: US 2005/0192243 A1
(43) Pub. Date: Sep. 1, 2005

(54) Neuromuscular blocking agents and antagonists thereof

(76) Inventor: John J. Savarese, Southbury, CT (US)

Correspondence Address: Schwegman, Lundberg, Woessner & Kluth, P.A., P.O. Box 2938, Minneapolis, MN, 55402, US

(21) Appl. No.: 10/975,197

(22) Filed: Oct. 28, 2004

Related U.S. Application Data
(60) Provisional application No. 60/515,048, filed on Oct. 28, 2003.

(51) Int. Cl. A61K 38/05; A61K 31/7076; A61K 31/4709; A61K 31/198

(52) U.S. Cl. 514/46; 514/18; 514/310; 514/562; 514/308

(57) ABSTRACT

The invention provides methods and kits for reversing the effects of ultra-short and intermediate duration halofumarate neuromuscular blocking agents that involve the use of cysteine and cysteine-like antagonists.

This application claims priority from U.S. Application Ser. No. 60/515,048 filed Oct. 28, 2003, which is incorporated herein by reference.

### FIELD OF THE INVENTION

The invention relates to ultra-short to intermediate acting neuromuscular blocking agents and methods for using and counteracting the effects of such neuromuscular blocking agents.

### BACKGROUND OF THE INVENTION

Administration of the long-acting neuromuscular blocker (NMB) d-tubocurarine (curare) (1a) to induce skeletal muscle relaxation during surgery and facilitate tracheal intubation maneuvers transformed the practice of anesthesia. Savarese et al., Pharmacology of Muscle Relaxants and Their Antagonists. In Anesthesia, 4th ed.; Miller, R. D., Ed.; Churchill Livingstone: N.Y., 1994; pp 417-488. Since that time a variety of semi-synthetic and synthetic neuromuscular blockers with varying durations of NMB (curare-like) activity became available in the clinic. Id.; Lee, Br. J. Anaesth. 2001, 87, 755-769; Rees et al., Annu. Rep. Med. Chem. 1996, 31, 41-50; Bevan, Pharmacol. Toxicol. 1994, 74, 3-9. Neuromuscular blockers are categorized both by their mechanism of action (nondepolarizing or depolarizing) and by their duration of action (ultra-short, short-, intermediate-, and long-acting). The maximum clinical duration of such neuromuscular blocker as defined by the FDA is the time for return to 25% of control in a twitch response test after a dose of twice the 95% effective dose (ED₉₅). This maximum duration time for an ultra-short neuromuscular blocker is 8 minutes, for a short neuromuscular blocker the duration is 20 minutes, for an intermediate neuromuscular blocker the duration time is 50 minutes and the duration time for a long acting neuromuscular blocker is greater than 50 minutes. See Bedford, Anesthesiology 1995, 82, 33A. Examples of these adjuncts to anesthesia include the long-acting agent metocurine (1b), the ultra-short-acting succinylcholine (2), the short-acting relaxant mivacurium (3), and the long-acting agent doxacurium (4).

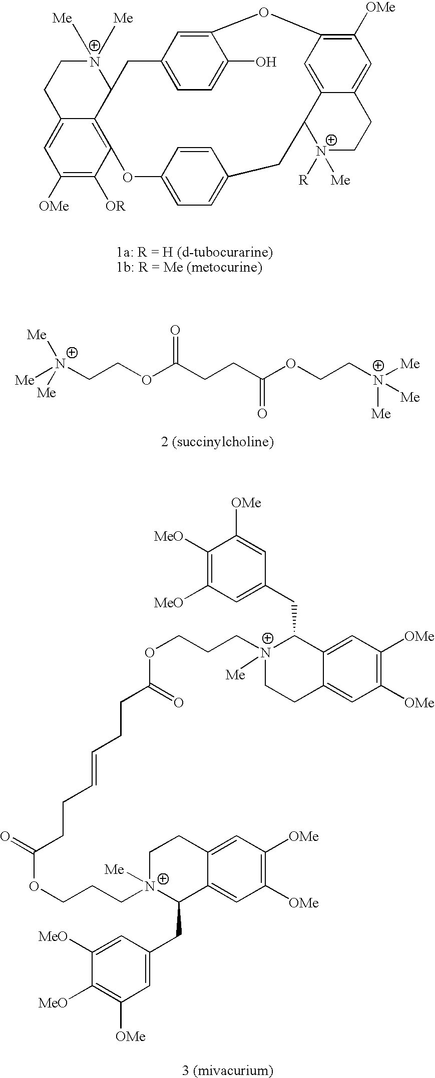

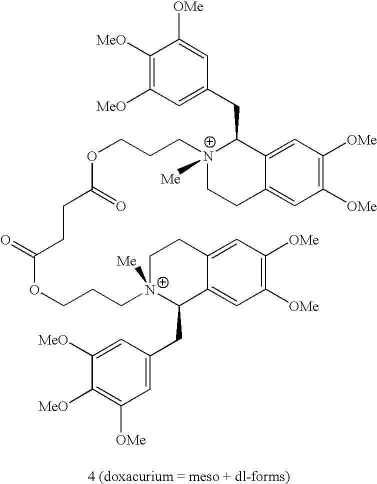

The benzyltetrahydroisoquinoline-based relaxants are nondepolarizing neuromuscular blockers. Succinylcholine (2) is a depolarizing agent. Depolarizing neuromuscular blockers are nicotinic acetylcholine receptor agonists and produce a number of unwanted side-effects associated with their mechanism of action. Naguib et al., Anesthesiology 2002, 96, 202-231; Mahajan, Curr. Anaesth. Crit. Care 1996, 7, 289-294; Belmont, Curr. Opin. Anesthesiol. 1995, 8, 362-366; Durant et al., Br. J. Anaesth. 1982, 54, 195-208. These untoward effects can, in rare instances, include anaphylaxis, hyperkalemia, malignant hyperthermia, and cardiac arrhythmias. More common side-effects of depolarizing neuromuscular blockers include fasciculations, severe muscle pain, increased intraocular pressure, and increased intragastric tension.

Nondepolarizing neuromuscular blockers are nicotinic acetylcholine receptor antagonists and are typically devoid of the side-effects associated with depolarizing relaxants. Although a variety of long-, intermediate-, and short-acting nondepolarizing neuromuscular blockers exist in the clinic, no ultra-short-acting nondepolarizing neuromuscular blocker is currently available. Consequently, when anesthesiologists require an ultra-short-acting neuromuscular blocker, they must choose succinylcholine. However succinylcholine can produce a number of unwanted side-effects, some of which can be life-threatening. Despite considerable research effort, there still exists no reliable substitute for succinylcholine in rapid sequence emergency intubations and treatment of laryngospasm. Hence, new ultra-short-acting neuromuscular blockers and methods for controlling the duration of ultra-short to intermediate neuromuscular blockers are needed.

### SUMMARY OF THE INVENTION

The invention provides methods, compositions and kits for controlling the maximum clinical duration of an ultra-short to intermediate halofumarate neuromuscular blockers. In one embodiment, the methods of the invention involve fast-acting agents that antagonize the neuromuscular blockade caused by administration of a halofumarate neuromuscular blocking agent. Agents that can antagonize the neuromuscular blockade caused by administration of a halofumarate neuromuscular blocking agent include cysteine, N-acetylcysteine, glutathione, as well as related cysteine analogs and combinations thereof.

Hence, the invention is directed to a therapeutic method comprising antagonizing the neuromuscular blockade caused by administration of a halofumarate neuromuscular blocking agent of formula I: 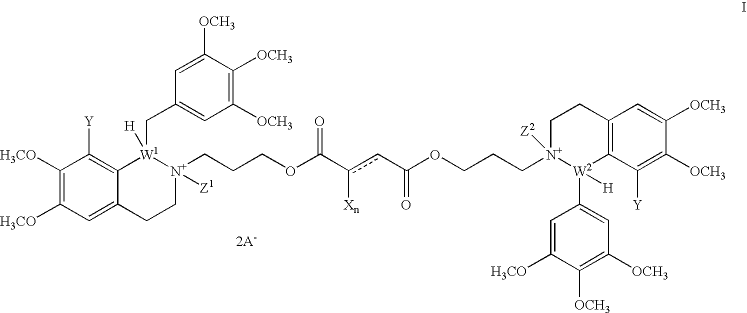
wherein:
X is halogen; n is an integer of 1 to 2;
Y is hydrogen or methoxy;
W¹ and W² are chiral carbon atoms;
Z¹ and Z² are methyl groups attached to chiral nitrogen atoms; and
A is a pharmaceutically acceptable anion.
   and wherein the method comprises administering an effective amount of a halofumarate neuromuscular blocking agent antagonist to a mammal subjected to said neuromuscular blockade. Examples of halofumarate neuromuscular blocking agent antagonists include cysteine, N-acetylcysteine, glutathione, homocysteine, methionine, S-adenosyl-methionine, penicillamine, a related cysteine analog, a combination thereof or a pharmaceutically acceptable salt thereof. In some embodiments, the antagonist is cysteine. In other embodiments, the antagonist is cysteine combined with a cysteine analog. For example, in some embodiments, the combination of cysteine and glutathione is particularly effective.

The invention further provides a kit that includes, separately packaged, (a) an amount of a halofumarate neuromuscular blocking agent sufficient to relax or block skeletal muscle activity, and (b) an amount of an antagonist to the halofumarate neuromuscular blocking agent effective to reverse the effects of the blocking agent on a mammal, with (c) instructions directing the user to employ the antagonist to reverse the effects of the blocking agent on a mammal to which the blocking agent is administered. Such an antagonist to a halofumarate neuromuscular blocking agent can, for example, be cysteine, N-acetylcysteine, glutathione, homocysteine, methionine, S-adenosyl-methionine, penicillamine, a combination thereof or pharmaceutically acceptable salts thereof in combination. In some embodiments, the antagonist is cysteine. In other embodiments, the antagonist is cysteine combined with a cysteine analog. For example, in some embodiments, the combination of cysteine and glutathione is particularly effective.

### DESCRIPTION OF THE DRAWINGS

FIG. 1 graphically summarizes the systolic blood pressure (▪), diastolic pressure (▴), mean arterial pressure (♦) and pulse rate (*), of GW 280430A-treated animals as a function of time after administration of the halofumarate neuromuscular blocking agent GW 280430A.

FIG. 2 graphically summarizes the systolic blood pressure (▪), diastolic pressure (▴), mean arterial pressure (*) and pulse rate (♦), of GW 280430A-treated animals as a function of time after administration of all tested dosages of cysteine. As illustrated, administration of cysteine has no significant affect on blood pressure or heart rate.

FIG. 3 provides an example of the rate of recovery after administration of a halofumarate neuromuscular blocking agent (GW 280430A) with (▪) and without (♦) administration of 5 mg/kg cysteine. As shown, administration of cysteine after the GW 280430A halofumarate neuromuscular blocking agent completely reverses the block in neuromuscular activity within about 100 sec after administration.

FIG. 4 provides an example of the rate of recovery after administration of a halofumarate neuromuscular blocking agent (GW 280430A) with (▪) and without (♦) administration of 5 mg/kg N-acetylcysteine. As shown, administration of N-acetylacysteine after the GW 280430A halofumarate neuromuscular blocking agent completely reverses the block in neuromuscular activity within about 165 seconds after administration.

FIG. 5 provides an example of the rate of recovery after administration of a halofumarate neuromuscular blocking agent (GW 280430A) with (▪) and without (♦) administration of 5 mg/kg glutathione. As shown, administration of glutathione after the GW 280430A halofumarate neuromuscular blocking agent completely reverses the block in neuromuscular activity within about 140 seconds after administration.

FIG. 6 provides a comparison of the speed of recovery of monkeys from the GW 280430A halofumarate neuromuscular blocking agent by cysteine (*), N-acetylcysteine (▴) and glutathione (▪). Recovery from the GW 280430A halofumarate neuromuscular blocking agent without administration of a cycteine-like molecule is also shown (♦).

FIG. 7 graphically summarizes the systolic blood pressure (▪), diastolic pressure (▴), mean arterial pressure (*) and pulse rate (♦), of GW 280430A-treated animals as a function of time after administration of 5 mg/Kg N-acetylcysteine.

FIG. 8 graphically summarizes the systolic blood pressure (▪), diastolic pressure (▴), mean arterial pressure (*) and pulse rate (♦), of GW 280430A-treated animals as a function of time after administration of 5 mg/Kg glutathione.

FIG. 9 provides a comparison of the speed of recovery of monkeys from the ultra-short duration GW 280430A and the intermediate duration 353044 halofumarate neuromuscular blocking agents with and without cysteine-like antagonist. The solid lines illustrate recovery of monkeys in the absence of antagonist administration from 0.50 mg/kg ultra-short GW 280430A (♦) or 0.10 mg/kg intermediate duration 353044 (*) halofumarate neuromuscular blocking agents. Recovery from 0.50 mg/kg GW 280430A ultra-short duration halofumarate neuromuscular blocking agent in the presence of 5 mg/kg cysteine is illustrated by the dashed line and the symbol ▪. Recovery of monkeys from 0.10 mg/kg of the 353044 intermediate duration halofumarate neuromuscular blocking agent in the presence of a combination of 10 mg/kg cysteine and 10/kg glutathione is illustrated by a dashed line and the symbol ▴. Note that 0.5 mg/kg of the ultra-short duration GW 280430A blocking agent is 5× of the 95% effective dose, whereas 0.1 mg/kg of the intermediate duration 353044 blocking agent is 2× of the 95% effective dose.

FIG. 10 provides a comparison of the speed of recovery of monkeys from the intermediate duration 353044 halofumarate neuromuscular blocking agents with and without cysteine-like antagonist. The graph illustrates recovery of monkeys from 0.10 mg/kg of the 353044 intermediate duration halofumarate neuromuscular blocking agent in the absence (♦) and presence (▪) of a combination of 10 mg/kg cysteine and 10/kg glutathione.

### DETAILED DESCRIPTION OF THE INVENTION

The present invention relates to novel methods for regulating the duration of halofumarate neuromuscular blocking agents of ultra-short to intermediate duration. Neuromuscular blocking agents can literally paralyze a patient for the time during which they are active. Hence, the use of neuromuscular blocking agents is restricted to situations where muscle relaxation is essential for effective treatment of a patient, for example, selected surgical procedures and those involving intubation of the trachea. Because paralysis can interfere with essential body functions (e.g. breathing) the physician selects a neuromuscular blocking agent that will be active for as long as needed but no more than is needed. Hence, ultra-short to intermediate duration neuromuscular blocking agents are frequently used to limit the duration of patient paralysis.

For example, when a breathing tube must be inserted into the trachea of a patient a neuromuscular blocking agent is used to relax the tracheal muscles and permit intubation. However, the neuromuscular blocking agent also relaxes the muscles of the chest, thereby causing the patient to stop breathing. The anesthesiologist must quickly insert the breathing tube into the patient's trachea and begin ventilation of the lungs. If the tube cannot be inserted quickly enough, the physician must intervene with some form of artificial resuscitation or the patient may suffer oxygen deprivation, and the associated tissue damage from lack of oxygen. Ultra-short duration neuromuscular blocking agents are often preferred in these circumstances because their duration is sufficient to permit intubation and the time period during which the patient will be paralyzed and unable to breathe on his or her own is short.

However, even ultra-short neuromuscular blocking agents are defined to have a maximum duration time of eight minutes, which is still a long time for a patient who cannot breathe without assistance. The invention provides a fast, reliable method for counteracting the effects of ultra-short to intermediate duration halofumarate neuromuscular blocking agents so that a patient will recover from the effects of such neuromuscular blocking agents within seconds of administering the appropriate antagonist.

The compounds of the invention are safer, more reliable and faster-acting against halofumarate neuromuscular blocking agents than currently available combinations of neuromuscular agents and antagonists. Moreover, the neuromuscular blockage can be counteracted with cysteine and cysteine-like molecules at any time, even just after administration of the blocking agent. This cannot be done with most currently available neuromuscular blocking agents and antagonists. An anesthesiologist must wait until a patient is spontaneously beginning to recover from the neuromuscular blocking agent before administering most currently available antagonists. The cysteine and cysteine-like antagonists of the invention also have substantially no side effects. The antagonists of the invention are compounds that are naturally found in the body and cause essentially no change in pulse rate, blood pressure or other indicators of cardiac function. The cysteine and cysteine-like antagonists of the invention act directly on halofumarate neuromuscular blocking agents and quickly convert them to inactive chemical derivatives. The cysteine and cysteine-like antagonists of the invention do not require inhibition of an important endogenous enzyme system, which is required by currently available antagonists of neuromuscular blocking agents such as neostignine, edrophonium and other cholinesterase inhibitors.

Halofumarate NMB Antagonists

According to the invention, cysteine, N-acetylcysteine, glutathione, related cysteine analogs and combinations thereof can be used to shorten the duration of halofumarate neuromuscular blocking agents. Such cysteine and cysteine-like molecules can react with and inactivate a halofumarate having formula Ia (halide substituent (X) between two carbonyl groups), shown below: 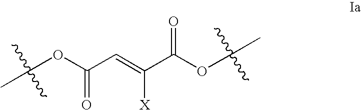
Upon reaction, for example, with cysteine, the halide is displaced and a thiazolidine derivative (Ib) forms. 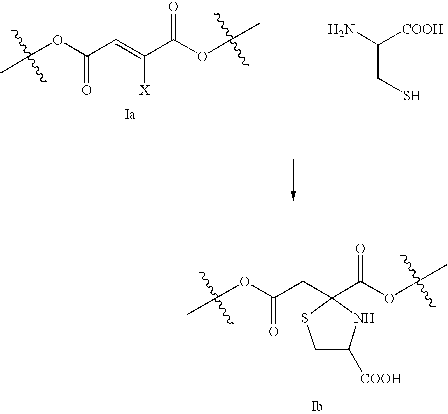
Formation of the inactive thiazolidine derivative (Ib) occurs quickly and the patient recovers from the effects of the halofumarate non-depolarizing neuromuscular blocking agent within about 30 seconds to about 300 seconds, often within about 30 seconds to about 180 seconds, or even within about 30 seconds to about 120 seconds.

The cysteine and cysteine-like small molecules that can be used in the methods of the invention include any substantially nontoxic compound having an amino and/or a sulfhydryl substituent that can displace the halide moiety in halofumarate that includes the structure of formula Ia. Examples of cysteine and cysteine-like small molecules that can be used include cysteine, N-acetylcysteine, glutathione, homocysteine, methionine, S-adenosyl-methionine, penicillamine and related cysteine analogs.

Ultra-Short to Intermediate Duration Halofumarate Neuromuscular Blocking Agents

According to the invention, the neuromuscular blocking activity of halofumarate non-depolarizing neuromuscular blocking agents with ultra-short to intermediate duration can be counteracted by administration of the cysteine and cysteine-like compounds as described herein. These halofumarate neuromuscular blocking agents have a duration time of about 5 to 15 minutes. However, when cysteine and/or cysteine-like compounds are administered the patient will recover from the effects of the halofumarate neuromuscular blocking agents within about 30 seconds to about 300 seconds, and in some embodiments within about 30 to about 180 seconds, or even within about 30 seconds to about 120 seconds. Hence, use of the methods, compositions and kits of the invention will provide increased safety over known antagonists for available ultra-short to intermediate duration blocking agents because of the speed at which they work and the absence of side effects.

Examples of halofumarate non-depolarizing neuromuscular blocking agents that can be inactivated by the cysteine and cysteine-like molecules provided herein include compounds described in U.S. Pat. No. 6,187,789, which is incorporated herein by reference. Other examples of halofumarate non-depolarizing neuromuscular blocking agents that can be inactivated by the cysteine and cysteine-like molecules provided herein include compounds of Formula I: 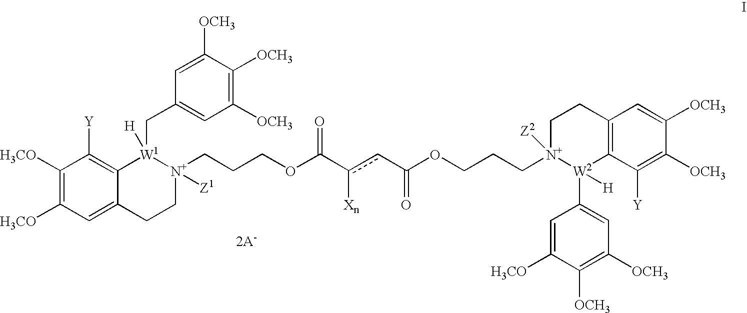
wherein: X is halogen; n is an integer of 1 to 2; Y is hydrogen or methoxy; W¹ and W² are chiral carbon atoms; Z¹ and Z² are methyl groups attached to chiral nitrogen atoms; and A is a pharmaceutically acceptable anion.

The compounds of Formula I contain two substituted isoquinolinium moieties connected by an aliphatic linker. The two substituted isoquinolinium moieties can be conveniently distinguished by referring to them as the “left hand ring structure” and the “right hand ring structure”, where the left hand ring structure contains W¹ and the right hand ring structure contains W². The aliphatic linker is the portion of the compound of Formula I denoted by the following Formula i. 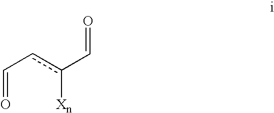
The combination of a solid and a dashed line (------) indicates that a double or single bond is present.

The halogen X can be any halogen, for example, iodine, choline, bromine or fluoro. In some embodiments, the compounds of Formula I used in the invention include those wherein X is chlorine, bromine or fluorine. Preferred halogen substitutions are monochloro, monobromo, monofluoro and difluoro.

The aliphatic linker portion of compounds of Formula I, as described by Formula i, comprises a butanedioate or butenedioate moiety. Suitably, compounds of Formula I wherein the aliphatic linker comprises a butenedioate moiety may exist in either the E or Z configuration or as mixtures of E and Z isomers. Preferably the butenedioate moiety of compounds of Formula I is a fumarate. The term fumarate as used herein refers to a butenedioate moiety wherein the two ester carbonyl groups are oriented trans to one another.

The compounds of Formula I contain four chiral centers. The carbon atoms (denoted as W¹ and W²) and each quaternary nitrogen atom in the isoquinolinium moieties are chiral. Each of the four chiral centers may independently exist in either the R or S configuration. Accordingly, as is apparent to those skilled in the art, each compound within Formula I may exist in sixteen distinct optical isomeric forms. The scope of the present invention extends to cover each and every isomer of the compounds of Formula I either individually or in admixture with other isomers, and all mixtures of such isomers. In some embodiments, W¹ is in the R configuration, the N attached to Z¹ is in the S configuration, W² is in either the R or S configuration, and the N attached to Z² is in either the R or S configuration. Preferably, W¹ is in the R configuration, and the N attached to Z¹ is in the S configuration.

In other embodiments, W² is in the R configuration, and the N attached to Z² is in either the R or S configuration. Compounds of Formula I wherein W¹ is in the R configuration, W² is in the S configuration, the N attached to Z¹ is in the S configuration and the N attached to Z² is in the R configuration are more preferred.

In one embodiment, the compound of Formula I has the following structure: 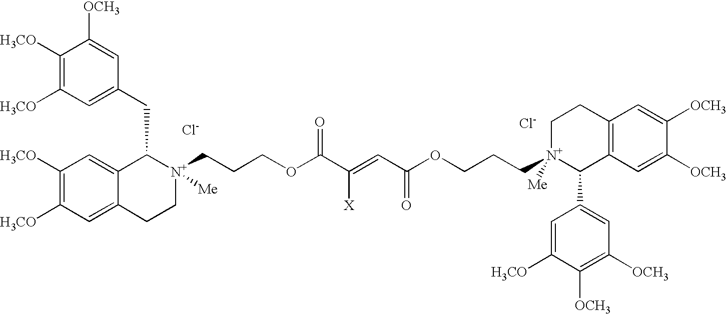
wherein: X is halogen. Preferably, X is chloride.

In another embodiment, the compound of Formula I has the following structure: 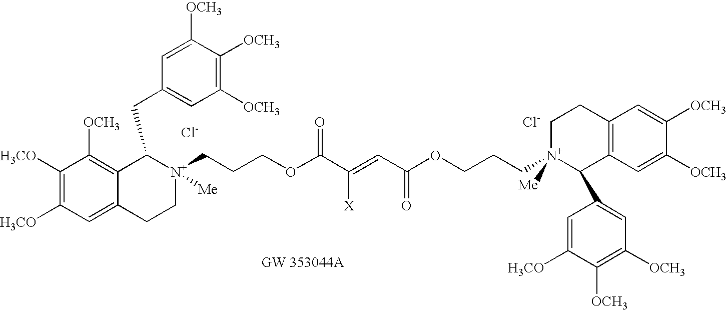
wherein X is halogen. In some embodiments, X is chloride.

In general, preferred compounds of Formula I include:
(Z)-2-Chloro-4-{3-[(1S,2R)-6,7-dimethoxy-2-methyl-1-(3,4,5-trimethoxyphenyl)-1,2,3,4-tetrahydro-2-isoquinolinio]propyl}-1-{3{(1R,2S)-6,7-dimethoxy-2-methyl-1-[(3,4,5-trimethoxyphenyl)methyl]-1,2,3,4-tetrahydro-2-isoquinolinio}propyl}-2-butenedioate dichloride (the GW280430A compound),
(Z)-2-Chloro-4-{3-[(1R,2S)-6,7-dimethoxy-2-methyl-1-(3,4,5-trimethoxyphenyl)-1,2,3,4-tetrahydro-2-isoquinolinio]propyl}-1-{3{(1R,2S)-2-methyl-6,7-dimethoxy-1-[(3,4,5-trimethoxyphenyl)methyl]-1,2,3,4-tetrahydro-2-isoquinolinio}propyl}-2-butenedioate dichloride (the GW353044A compound),
2,2-Difluoro-4-{3-[(1S,2R)-6,7-dimethoxy-2-methyl-1-(3,4,5-trimethoxyphenyl)-1,2,3,4-tetrahydro-2-isoquinolinio]propyl}1-{3-{(1R,2S)-6,7-dimethoxy-2-methyl-1-[(3,4,5-trimethoxyphenyl)methyl]-1,2,3,4-tetrahydro-2-isoquinolinio}propyl}-butanedioate dichloride,
(Z)4-{3-[(1S,2R)-6,7-Dimethoxy-2-methyl-1-(3,4,5-trimethoxyphenyl)-1,2,3,4-tetrahydro-2-isoquinoliniolpropyl}-1-{3-{(1R,2S)-6,7-dimethoxy-2-methyl-1-[(3,4,5-trimethoxyphenyl)methyl]-1,2,3,4-tetrahydro-2-isoquinolinio}propyl}-2-fluoro-2-butenedioate dichloride and
2,2-Difluoro-4-{3-[(1S,2R)-6,7-dimethoxy-2-methyl-1-(3,4,5-trimethoxyphenyl)-1,2,3,4-tetrahydro-2-isoquinolinio]propyl}-1-{3-{(1R,2S)-2-methyl-6,7,8-trimethoxy-1-[(3,4,5-trimethoxyphenyl)methyl]-1,2,3,4-tetrahydro-2-isoquinolinio}propyl}butanedioate dichloride.

The halofumarate compounds described herein can be made as described in U.S. Pat. No. 6,187,789 and Boros et al., J. Med. Chem. 46:2502-15 (2003), which are incorporated herein by reference.

The pharmacological activity of the compounds of the invention resides in the cation. Hence, the nature of the anion A⁻ is relatively unimportant. However, for therapeutic purposes, A⁻ is preferably pharmaceutically acceptable to the recipient of the compounds. Examples of pharmaceutically acceptable anions include iodide, mesylate, tosylate, bromide, chloride, hydrogen sulphate, sulphate⁻², phosphate⁻³, hydrogen phosphates, acetate, besylate, succinate⁻², maleate, naphthalenesulphonate and propionate. Both pharmaceutically acceptable salts and salts that are not thus acceptable may be useful for isolating and/or purifying the compounds of the invention. The pharmaceutically unacceptable salts may also be useful in that they may be converted into acceptable salts by techniques available in the art.

The compounds of Formula I are used as neuromuscular blocking agents during surgery, for intubation of the trachea or during electroshock therapy. They may be administered parenterally, e.g., by intramuscular or intravenous injection of a solution.

Methods of Use

The present invention also provides a method for reversing muscle relaxation caused by compounds of Formula I in a mammal. Such methods include administering to the mammal an amount of a cysteine or cysteine-like molecule that is effective for reversing the neuromuscular block produced by a compound of Formula I. The dosage for each subject may vary, however, a suitable intravenous amount or dosage of the compounds of Formula I to obtain paralysis in mammals would be 0.01 to 20.0 mg/kg of body weight, or about 0.02 to 2.0 mg/kg of body weight, the above being based on the weight of the di-cation which is the active ingredient. The dosage for intramuscular administration is two to eight times the intravenous dose.

In a further aspect, the present invention provides compounds of Formula I with an effective amount of cysteine or a cysteine-like molecule for use in therapy, for example to induce neuromuscular blockade in surgery or for intubation of the trachea, and then to reverse the neuromuscular blockade. The present invention also provides the use of cysteine or a cysteine-like molecule with or without a compound of Formula I in the manufacture of a medicament for reversing neuromuscular blockade in a mammal, including in a human.

While it is possible for the cysteine, cysteine-like molecules and/or compounds of Formula I to be administered as the bulk active chemicals, it is preferred to present them in the form of a pharmaceutical formulation for parenteral administration. Accordingly, the present invention provides a pharmaceutical formulation which comprises a cysteine or cysteine-like molecule, as hereinbefore defined and a pharmaceutically acceptable carrier.

Where the pharmaceutical formulation is for parenteral administration, the formulation may be an aqueous or non-aqueous solution or mixture of liquids, which may contain bacteriostatic agents, antioxidants, buffers or other pharmaceutically acceptable additives. Alternatively the compounds may be presented as lyophilized solids for reconstitution with water (for injection) or dextrose or saline solutions. Such formulations are normally presented in unit dosage forms such as ampoules or disposable injection devices. They may also be presented in multi-dose forms such as a bottle from which the appropriate dose may be withdrawn. All such formulations should be sterile.

A suitable dose to obtain a neuromuscular block for adult humans (150 lbs. or 70 kg) is about 0.1 mg to about 500 mg, or in some embodiments about 0.5 mg to about 150 mg, or in other embodiments about 3.5 mg to about 50 mg. Thus a suitable pharmaceutical parenteral preparation for administration to humans will preferably contain 0.1 to 50 mg/ml of the compounds of Formula I in solution or multiples thereof for multi-dose vials. A suitable dose of cysteine or a cysteine-like molecule to reverse a neuromuscular block in adult humans (150 lbs. or 70 kg) is about 50 mg to about 2000 mg or about 150 to about 750 mg. Thus a suitable pharmaceutical parenteral preparation for administration to humans will preferably contain 0.1 to 100 mg/ml of cysteine or a cysteine-like molecule in solution or multiples thereof for multi-dose vials.

A simple formulation is a solution of cysteine or a cysteine-like molecule in sterile water or saline solution. This may be prepared by dissolving the compound in pyrogen-free water or saline, with or without a preservative and sterilizing the solution. Alternatively, it may be prepared by dissolving the sterile compound in pyrogen-free, sterile water or a sterile physiological saline solution under aseptic conditions. Particularly preferred formulations have a pH of about 2.0 to 5.0. The cysteine or cysteine molecules of the invention may also be administered as a rapid intravenous bolus over about 5 seconds to about 15 seconds or, alternatively, as a slower infusion over about 1 to about 2 minutes of a saline solution, e.g., Ringer's solution in drip form.

The compounds may also be administered in other solvents (usually as a mixed solvent with water) such as alcohol, polyethylene glycol and dimethylsulphoxide. They may also be administered intravenously or intramuscularly (as a drip if required) as a suspension or solution.

The following examples further illustrate but are not intended to limit the invention.

### EXAMPLE 1

### Methods of Administering an Ultra-Short Halofumarate Neuromuscular Blocking Agent

Rhesus monkeys were anesthetized with ketamine (5 mg/Kg) and thiopental (5 mg/Kg) given intramuscularly or intravenously. Anesthesia was maintained with a mixture of halothane (1.5%), nitrous oxide (60%) and oxygen (40%). The common peroneal nerve was stimulated supramaximally with square wave pulses of 0.2 m sec duration at a rate of 0.15 Hz. Twitch contractions were recorded via the tendon of the extensor digitorum muscle.

In all animals, the trachea was intubated and ventilation was controlled at 12-15 ml/kg, 18-24 breaths per minute. A peripheral vein and artery were cannulated for drug administration and for recording or arterial pressure, respectively. Halofumarate neuromuscular blocking agent having the following structure, wherein X is Cl, was administered intravenously. 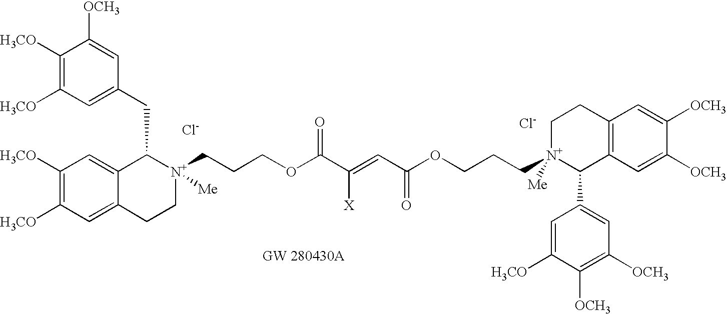
Thereafter, cysteine, N-acetylcysteine or glutathione was administered to test animals at selected dosages.

### EXAMPLE 2

### Cysteine Provides Fast Recovery from Halofumarate Neuromuscular Block

Rhesus monkeys were anesthetized and treated with a halofumarate neuromuscular blocking agent as described in Example 1.

Table 1 shows how quickly the four Rhesus monkeys tested (Skye, Impy, Morgan, and Count) recovered from administration of the halofumarate neuromuscular blocking agent (GW 280430A or “430A”) with and without cysteine administration. Several tests were generally run (#1, #2, #3, etc.) with time in between each test so that the animal could recover.

TABLE 1
Recovery from 430A with or without Cysteine

| Skye - 12.1 kg | | #1 - 0.05 mg/kg | #2 - 0.05 mg/kg 430A | | #3 - 0.05 mg/kg |
|---|---|---|---|---|---|
| Recovery | | 430A | & cysteine 2 mg/kg | | 430A |
| 5% | | 9 sec | 10 | | N/A |
| 25% | | 51 sec | 35 | | 143 |
| 75% | | 186 sec | 100 | | 265 |
| 95% | | 372 sec | 218 | | 368 |
| | 25-75% | 135 sec | 65 | | 122 |
| | 5-95% | 363 sec | 208 | sec | |
| Impy - 12.5 kg | | #1 - 0.05 mg/kg | #2 - 0.05 mg/kg 430A | | #3 - 0.05 mg/kg |
| Recovery | | 430A | & cysteine 5 mg/kg | | 430A |
| 5% | | 30 | 6 | | N/A |
| 25% | | 63 | 33 | | N/A |
| 75% | | 120 | 48 | | 45 |
| 95% | | 171 | 96 | | 78 |
| | 25-75% | 57 | 15 | | #VALUE! |
| | 5-95% | 141 | 90 | | #VALUE! |
| Morgan 12.4 kg | | #1 - 0.05 mg/kg | #3 - 0.1 mg/kg & | | #2 - 0.01 mg/kg |
| Recovery | | 430A | cysteine 20 mg/kg | | 430A |
| 5% | | N/A | 24 | | 30 |
| 25% | | 40 | 42 | | 42 |
| 75% | | 143 | 66 | | 186 |
| 95% | | 233 | 168 | | 312 |
| | 25-75% | 103 | 24 | | 144 |
| | 5-95% | | 144 | | 282 |

| Count 13.1 kg | | #4 - 0.2 mg/kg & |
|---|---|---|
| Recovery | #3 - 0.2 mg/kg | cysteine 4 mg/kg |

| | | | |
|---|---|---|---|
| 5% | | 9 | 9 |
| 25% | | 24 | 15 |
| 75% | | 57 | 30 |
| 95% | | 84 | 36 |
| | 25-75% | 33 | 15 |
| | 5-95% | 75 | 27 |

Table 2 illustrates that even after 3 administrations of the halofumarate neuromuscular blocking agent (GW 280430A or “430A”), the animals still recover quickly when cysteine is administered.

TABLE 2
Recovery from 430A with Cysteine

| Recovery | |
|---|---|
| | #4 - 0.05 mg/kg 430A & cysteine 5 mg/kg |
| 5% | 15 |
| 25% | 39 |
| 75% | 102 |
| 95% | 252 |
| 25-75% | 63 |
| 5-95% | 237 |
| | #4 - 0.05 mg/kg & cysteine 10 mg/kg |
| 5% | 18 |
| 25% | 51 |
| 75% | 66 |
| 95% | 114 |
| 25-75% | 15 |
| 5-95% | 96 |

Table 3 provides mean recovery values for the four Rhesus monkeys tested (Skye, Impy, Morgan, and Count) after administration of the halofumarate neuromuscular blocking agent (GW 280430A or “430A”) with and without cysteine administration.

TABLE 3
Mean Recovery from 430A with Cysteine

| Means = Skye, | | | |
|---|---|---|---|
| Impy & Count | | | 0.05 mg/kg 430A & cysteine 4 or |
| Recovery | | Baseline | 5 mg/kg |
| 5% | | 16 | 10 |
| 25% | | 46 | 29 |
| 75% | | 121 | 60 |
| 95% | | 209 | 125 |
| | 25-75% | 75 | 31 |
| | 5-95% | 193 | 115 |
| Skye - | | | |
| 11.6 kg | | | |
| Recovery | | Baseline | 0.2 mg/kg 430A & cysteine 10.0 mg/kg |
| 5% | | 24 | 6 |
| 25% | | 72 | 18 |
| 75% | | 192 | 36 |
| 95% | | 348 | 96 |
| | 25-75% | 120 | 18 |
| | 5-95% | 324 | 90 |
| Buck = 13.2 | | | |
| Recovery | | Baseline | 0.2 mg/kg 430A & cysteine 10.0 mg/kg |
| 5% | | 12 | 6 |
| 25% | | 48 | 24 |
| 75% | | 102 | 26 |
| 95% | | 126 | 48 |
| | 25-75% | 54 | 2 |
| | 5-95% | 114 | 42 |
| Means = Skye, | | | |
| Impy and Buck | | | |
| Recovery | | Baseline | Cysteine 10 mg/kg |
| 5% | | 22 | 10 |
| 25% | | 61 | 31 |
| 75% | | 138 | 43 |
| 95% | | 215 | 86 |
| | 25-75% | 77 | 12 |
| | 5-95% | 193 | 76 |

### EXAMPLE 3

### Halofumarate Neuromuscular Blocking Agents and Cysteine Administration have Essentially No Side Effects

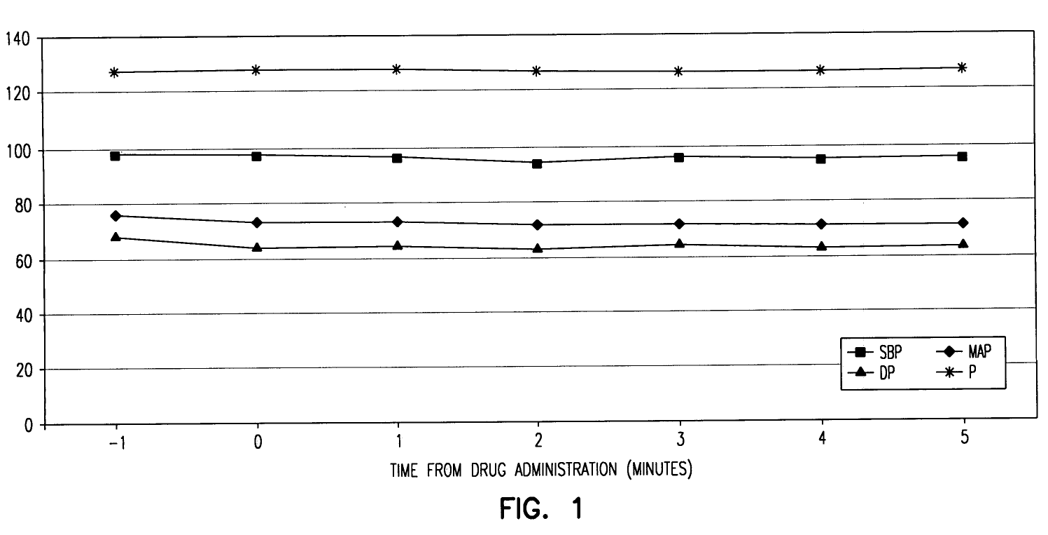

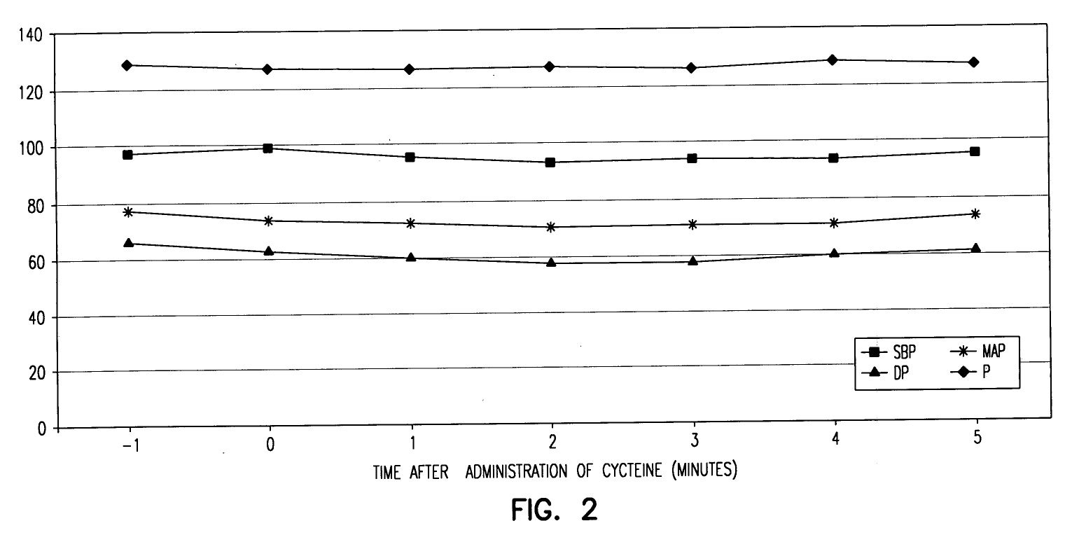

Rhesus monkeys were anesthetized and treated with a halofumarate neuromuscular blocking agent as described in Example 1. FIGS. 1 and 2 provide mean values for the cardiovascular effects of administration of the halofumarate neuromuscular blocking agent (GW 280430A or “430A”) over time (min.) with and without cysteine for the four Rhesus monkeys tested (Skye, Impy, Morgan, and Count).

FIG. 1 graphically summarizes the systolic blood pressure (▪), diastolic pressure (▴), mean arterial pressure (♦) and pulse rate (*), of GW 280430A-treated animals as a function of time after administration of the halofumarate neuromuscular blocking agent GW 280430A. FIG. 2 graphically summarizes the systolic blood pressure (▪), diastolic pressure (▴), mean arterial pressure (X) and pulse rate (⋄), of GW 280430A-treated animals as a function of time after administration of all tested dosages of cysteine. As illustrated, administration of cysteine has no significant affect on blood pressure or heart rate in the therapeutic range (5-10 mg/Kg). The highest dosage tested was 20 mg/kg, which was significantly more than needed to antagonize the effects of the neuromuscular blocker. Even at this very high dosage, cysteine caused no negative side effects.

### EXAMPLE 4

### Glutathione and N-Acetylcysteine Also Provide Fast Recovery from Halofumarate Neuromuscular Block

Rhesus monkeys were anesthetized and treated with a halofumarate neuromuscular blocking agent as described in Example 1.

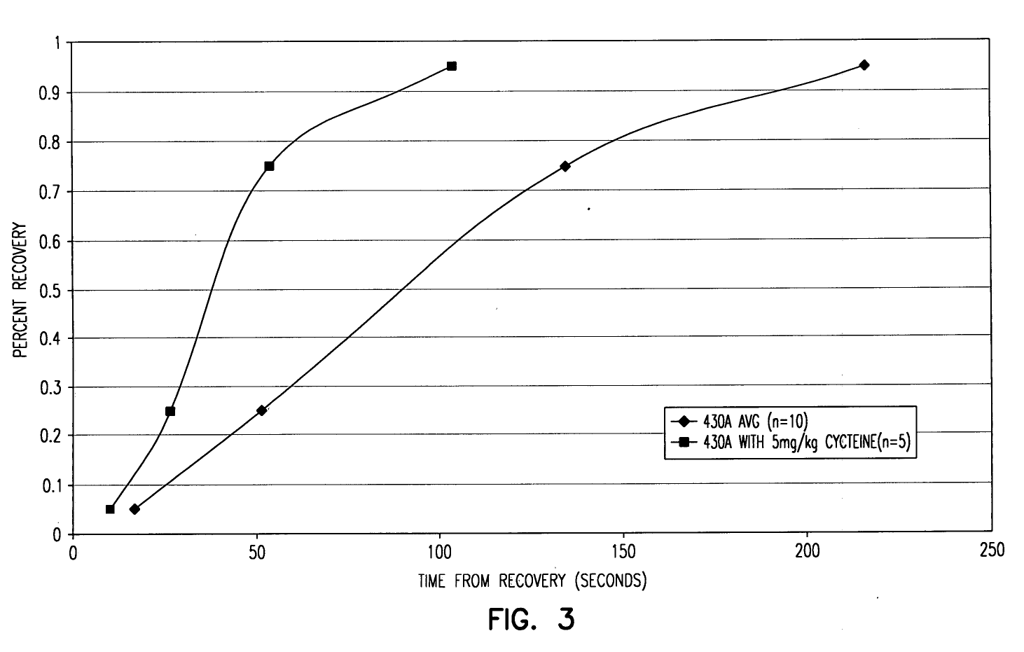

FIG. 3 provides an example of the rate of recovery after administration of a halofumarate neuromuscular blocking agent (GW 280430A) with administration of 5 mg/kg cysteine. As shown, administration of cysteine after the GW 280430A halofumarate neuromuscular blocking agent completely reverses the block in neuromuscular activity within about 100 sec after administration.

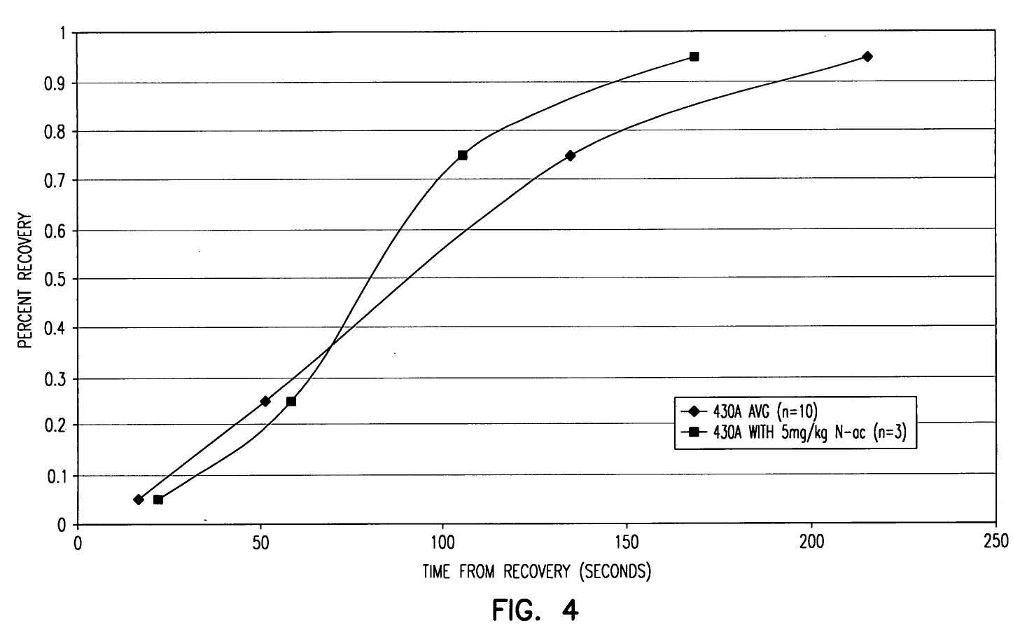

FIG. 4 provides an example of the rate of recovery after administration of a halofumarate neuromuscular blocking agent (GW 280430A) with administration of 5 mg/kg N-acetylcysteine. As shown, administration of N-acetylacysteine after the GW 280430A halofumarate neuromuscular blocking agent completely reverses the block in neuromuscular activity within about 165 seconds after administration.

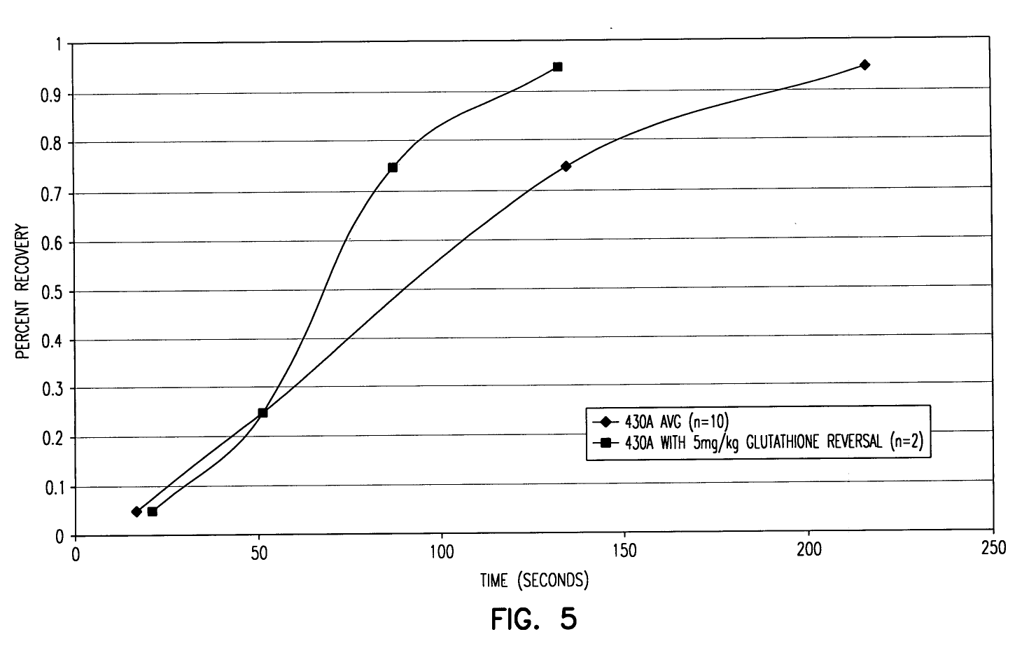

FIG. 5 provides an example of the rate of recovery after administration of a halofumarate neuromuscular blocking agent (GW 280430A) with administration of 5 mg/kg glutathione. As shown, administration of glutathione after the GW 280430A halofumarate neuromuscular blocking agent completely reverses the block in neuromuscular activity within about 140 seconds after administration.

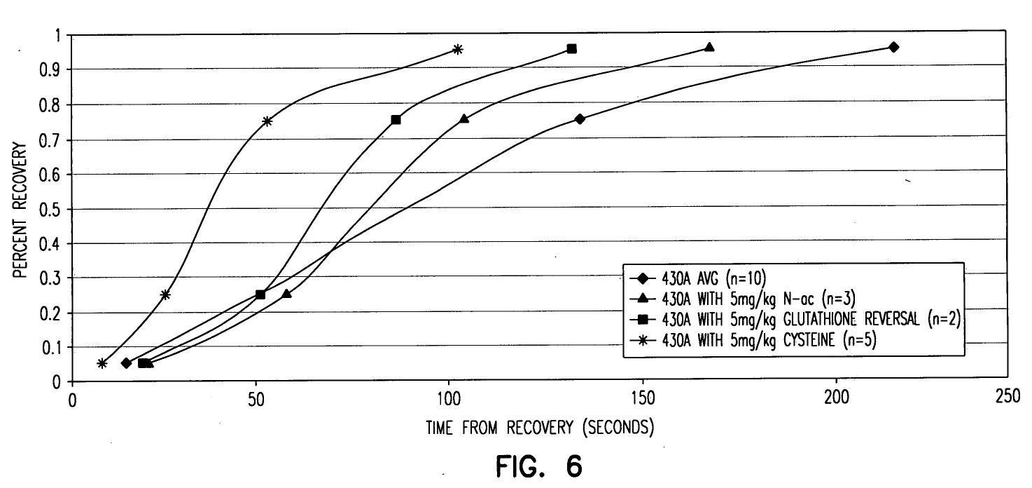

A comparison of the speed of recovery of monkeys from the GW 280430A halofumarate neuromuscular blocking agent by cysteine, N-acetylcysteine and glutathione is provided in FIG. 6.

### EXAMPLE 5

### Glutathione and N-Acetylcysteine Have Essentially No Side Effects

Rhesus monkeys were anesthetized and treated with the GW 280430A halofumarate neuromuscular blocking agent as described in Example 1. Glutathione or N-acetylcysteine (5 mg/kg) was administered as an antagonist to GW 280430A, instead of the cysteine.

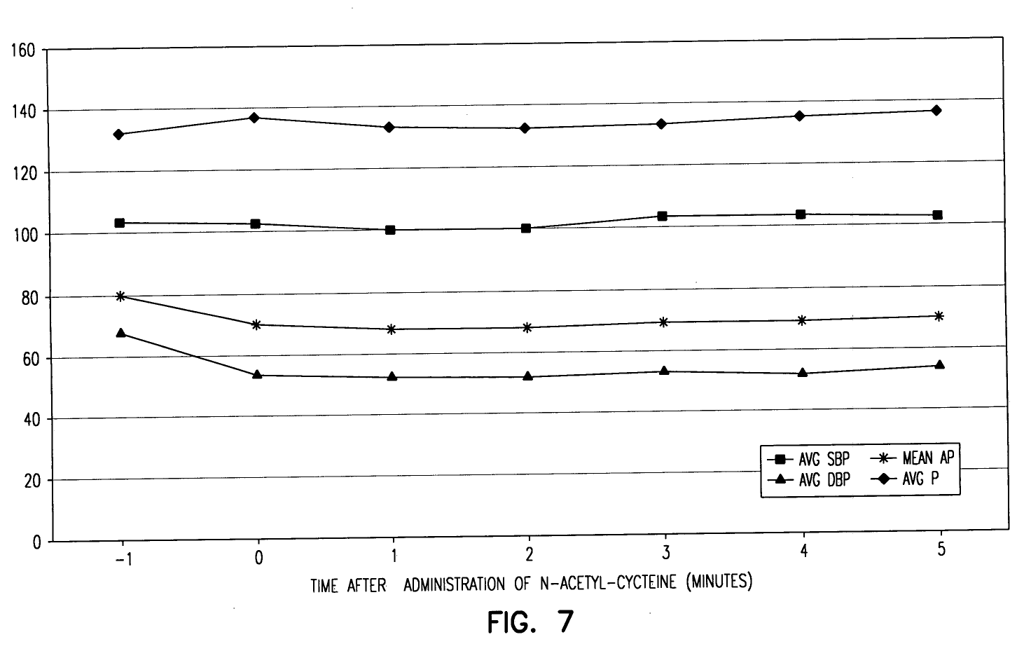

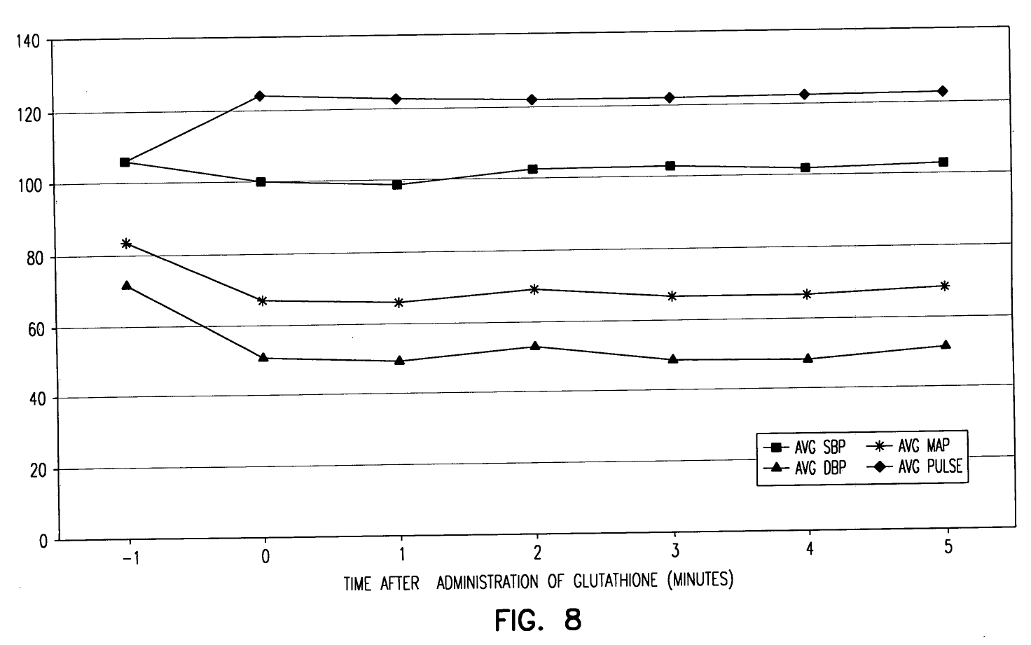

FIG. 7 graphically summarizes the systolic blood pressure (▪), diastolic pressure (▴), mean arterial pressure (X) and pulse rate (⋄), of GW 280430A-treated animals as a function of time after administration of 5 mg/Kg N-acetylcysteine. FIG. 8 graphically summarizes the systolic blood pressure (▪), diastolic pressure (▴), mean arterial pressure (X) and pulse rate (⋄), of GW 280430A-treated animals as a function of time after administration of 5 mg/Kg glutathione. As illustrated, administration of N-acetylcysteine and glutathione after administration of the GW 280430A halofumarate neuromuscular blocking agent has no significant affect on blood pressure or heart rate.

### EXAMPLE 6

### Cysteine-Like Molecules also Reverse Intermediate Duration Halofumarate Neuromuscular Blocking Agents

Rhesus monkeys were anesthetized with ketainine (5 mg/Kg) and thiopental (5 mg/Kg) given intramuscularly or intravenously. Anesthesia was maintained with a mixture of halothane (1.5%), nitrous oxide (60%) and oxygen (40%). The common peroneal nerve was stimulated supramaximally with square wave pulses of 0.2 m sec duration at a rate of 0.15 Hz. Twitch contractions were recorded via the tendon of the extensor digitorum muscle.

In all animals, the trachea was intubated and ventilation was controlled at 12-15 ml/kg, 18-24 breaths per minute. A peripheral vein and artery were cannulated for drug administration and for recording of arterial pressure, respectively. A dosage of 0.1 mg/kg of the 353044 intermediate duration halofumarate neuromuscular blocking agent was administered intravenously. The 353044 intermediate duration halofumarate neuromuscular blocking agent has the following structure, wherein X is Cl.

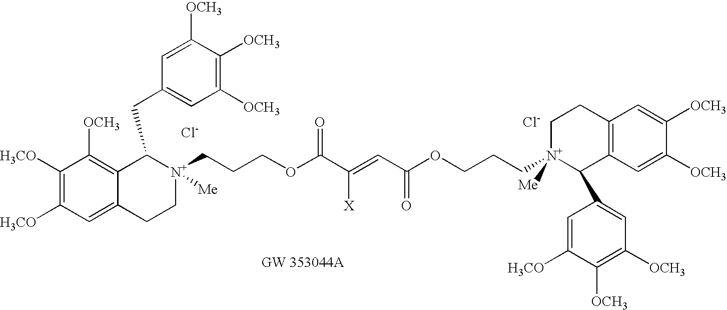

For comparison, 0.5 mg/kg of the ultra-short 280430A halofumarate neuromuscular blocking agent was administered to a separate set of animals. Thereafter, 5 mg/kg cysteine was administered to test animals that had received the ultra-short duration 280430A halofumarate neuromuscular blocking agent. Animals that had received the 353044 intermediate duration halofumarate neuromuscular blocking agent, then received a combination of 10 mg/kg and 10 mg/kg glutathione. Control animals received no cysteine or glutathione.

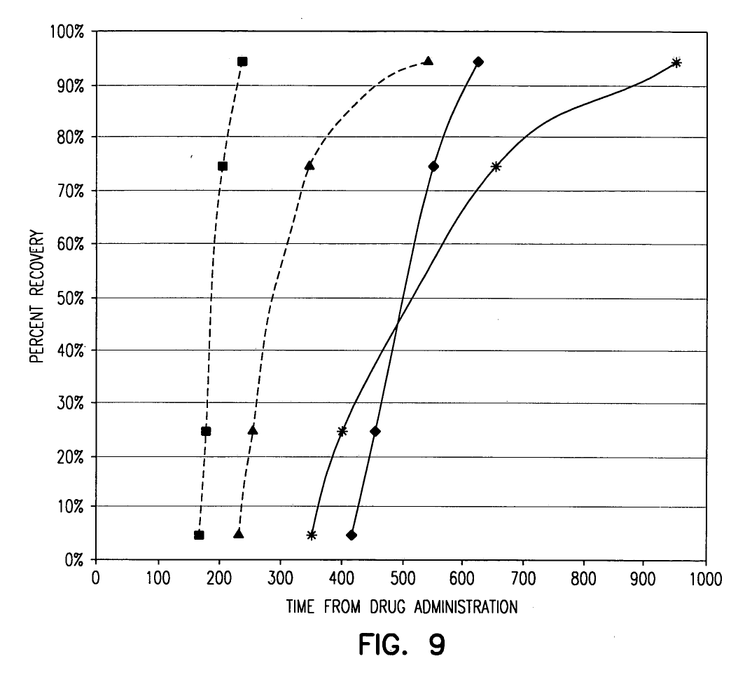

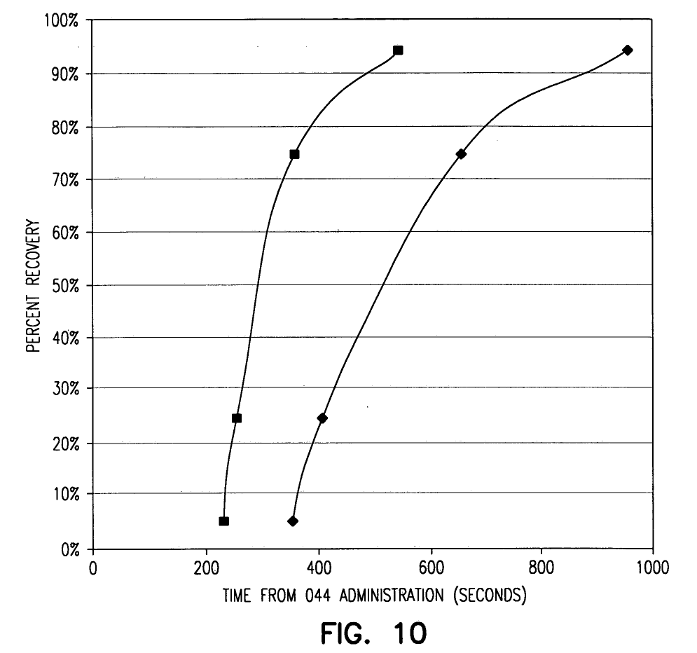

The results of these experiments are provided in FIG. 9 and 10. As shown in FIG. 9, monkeys receiving only the ultra-short duration GW 280430A blocking agent recovered faster than those receiving the intermediate duration 353044 halofumarate neuromuscular blocking agent (FIG. 9, compare solid lines showing recovery from the GW 280430A (♦) or 353044 (X) halofumarate neuromuscular blocking agent). In the presence of cysteine, recovery from 0.50 mg/kg GW 280430A (▪) was again faster than recovery from 0.10 mg/kg 353044 (▴) intermediate duration halofumarate neuromuscular blocking agent in the presence of a combination of cysteine and glutathione. Note that the cysteine-induced recovery from the GW 280430A block was faster even though a higher dose of the GW 280430A agent was used (5× of the 95% effective dose for GW 280430A; the 353044 dose was 2× of the 95% effective dose). Note also that the amount of cysteine used for reversing the GW 280430A block (5 mg/kg cysteine) was less than that used for reversing the 353044 block (10 mg/kg cysteine plus 10 mg/kg glutathione). FIG. 10 provides a comparison of the speed of recovery of monkeys from the intermediate duration 353044 halofumarate neuromuscular blocking agents with and without the antagonist combination (cysteine+glutathione). As illustrated, the combination of cysteine and glutathione effectively reverses the effects of the intermediate duration 353044 blocking agent within about 300 seconds.

Thus, the effects of both ultra-short and intermediate duration halofumarate neuromuscular blocking agents can readily be reversed by treatment with cysteine and/or cysteine analogs.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A therapeutic method comprising antagonizing the neuromuscular blockade caused by administration of a halofumarate neuromuscular blocking agent of formula I: 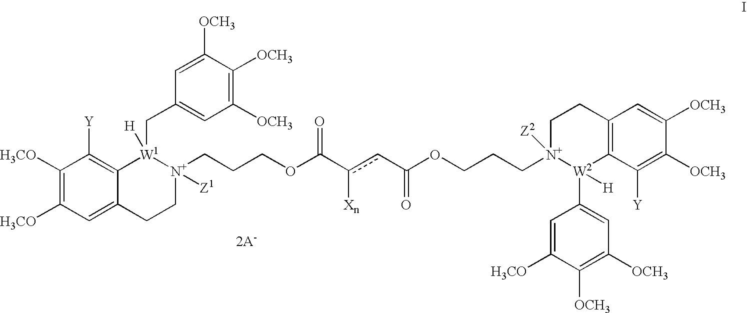
wherein:
X is halogen;
n is an integer of 1 to 2;
Y is hydrogen or methoxy;
W¹ and W² are chiral carbon atoms;
Z¹ and Z² are methyl groups attached to chiral nitrogen atoms; and
A is a pharmaceutically acceptable anion.
and wherein the method comprises administering an effective antagonizing amount of cysteine, a cysteine analog or pharmaceutically acceptable salts thereof to a mammal subjected to the neuromuscular blockade.

2. The method of claim 1, wherein the cysteine analog is N-acetylcysteine, glutathione, homocysteine, methionine, S-adenosyl-methionine, penicillamine, a combination thereof or a pharmaceutically acceptable salt thereof.

3. The method of claim 1, wherein the mammal is also subjected to general anesthesia.

4. The method of claim 1, wherein said compound of formula I is: 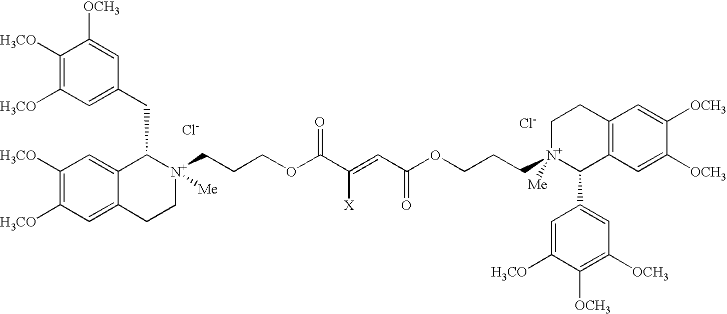
wherein X is halogen.

5. The method of claim 1, wherein said compound of formula I is: 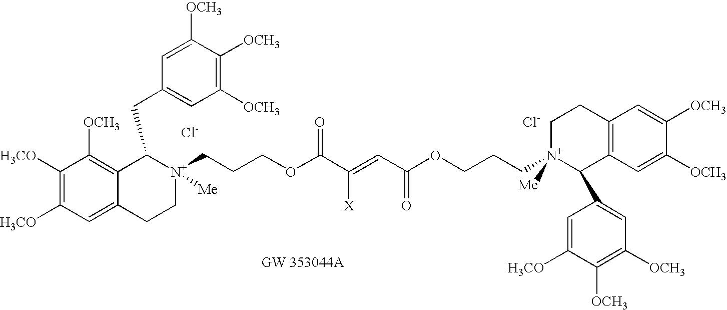
wherein X is chloride.

6. The method of claim 1, wherein X is chloride.

7. The method of claim 1, wherein cysteine, N-acetylcysteine, glutathione, homocysteine, methionine, S-adenosyl-methionine, penicillamine, a combination thereof or pharmaceutically acceptable salts thereof are administered intravenously, in combination with a pharmaceutically acceptable liquid carrier.

8. The method of claim 1, wherein cysteine, N-acetylcysteine, glutathione, a combination thereof or a pharmaceutically acceptable salt thereof is administered.

9. The method of claim 1, wherein a combination of cysteine and glutathione is administered.

10. The method of claim 1, wherein cysteine is administered.

11. The method of claim 1, wherein a cysteine or cysteine analog dosage of about 0.01 mg/kg to about 50 mg/kg is administered.

12. The method of claim 1, wherein the mammal is a domestic animal.

13. The method of claim 1, wherein the mammal is a human.

14. A therapeutic method comprising antagonizing a neuromuscular blockade caused by administration of a halofumarate neuromuscular blocking agent of the following formula: 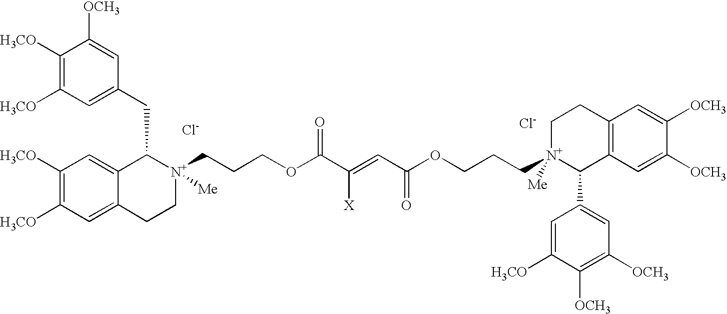
wherein:
X is halogen; and
wherein the method comprises administering an effective antagonizing amount of cysteine, N-acetylcysteine, glutathione, homocysteine, methionine, S-adenosyl-methionine, penicillamine or pharmaceutically acceptable salts thereof to a mammal subjected to the neuromuscular blockade.

15. A therapeutic method comprising antagonizing a neuromuscular blockade caused by administration of a halofumarate neuromuscular blocking agent of the following formula: 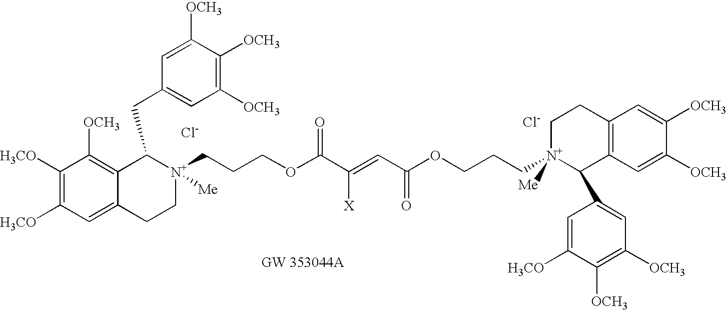
wherein:
X is halogen; and
wherein the method comprises administering an effective antagonizing amount of cysteine, N-acetylcysteine, glutathione, homocysteine, methionine, S-adenosyl-methionine, penicillamine or pharmaceutically acceptable salts thereof to a mammal subjected to the neuromuscular blockade.

16. A kit comprising, separately packaged, (a) an amount of a halofumarate neuromuscular blocking agent of Formula I: 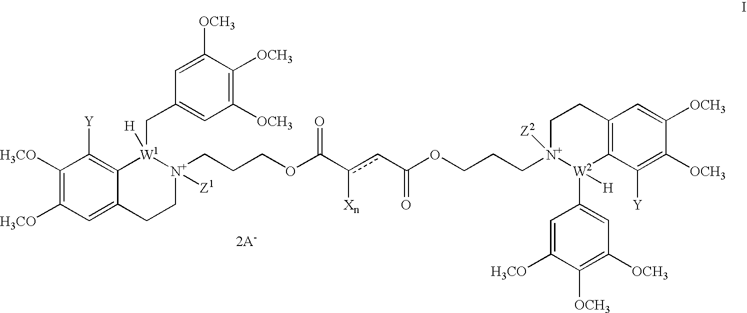
wherein:
X is halogen; n is an integer of 1 to 2;
Y is hydrogen or methoxy;
W¹ and W² are chiral carbon atoms;
Z¹ and Z² are methyl groups attached to chiral nitrogen atoms; and
A is a pharmaceutically acceptable anion;
(b) an effective amount of an antagonist to the halofumarate neuromuscular blocking agent, and
(c) instructions directing the user to employ the antagonist to reverse the effects of the blocking agent on a mammal to which the blocking agent is administered; wherein the antagonist is cysteine, N-acetylcysteine, glutathione, homocysteine, methionine, S-adenosyl-methionine, penicillamine, a combination thereof or pharmaceutically acceptable salts thereof.

17. The kit of claim 16, wherein the halofumarate neuromuscular blocking agent is: 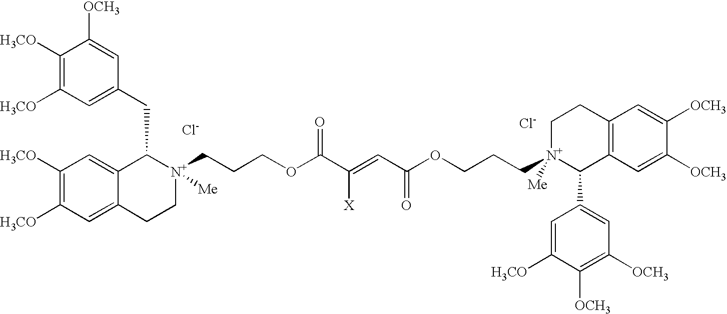
wherein X is halogen.

18. The kit of claim 16, wherein the halofumarate neuromuscular blocking agent is: 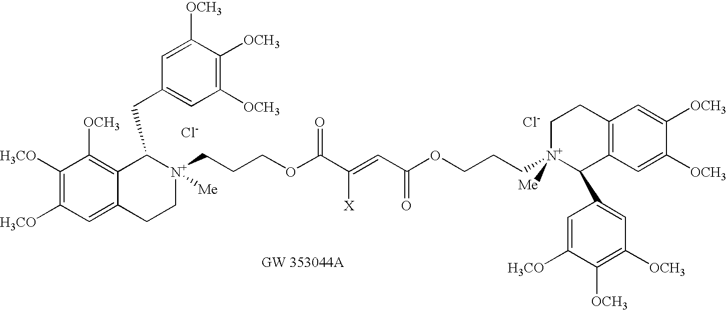
wherein X is halogen.

19. The kit of claim 16, wherein X is chloride.

20. The kit of claim 16, wherein the antagonist is formulated to be administered intravenously, in combination with a pharmaceutically acceptable liquid carrier.

21. The kit of claim 16, wherein the antagonist is cysteine, N-acetylcysteine, glutathione, a combination thereof or a pharmaceutically acceptable salt thereof.

22. The kit of claim 16, wherein the antagonist is a combination of cysteine and glutathione.

23. The kit of claim 16, wherein the antagonist is cysteine.
